(19) United States
(12) Patent Application Publication
(10) Pub. No.: US 2010/0048731 A1
(43) Pub. Date: Feb. 25, 2010

(54) Hydroxylated Long-Chain Resveratrol Derivatives Useful as Neurotrophic Agents

(75) Inventors: Bang Luu, Strasbourg (FR); Frédérique Hauss, Niederlauterbach (FR); Djalil Coowar, Strasbourg (FR); Jiawei Liu, Strasbourg (FR); Paul Heuschling, Garnich (LU); Eleonora Morga, Luxembourg (LU); Luc Grandbarbe, Metz (FR); Alessandro Michelucci, Luxembourg (LU)

Correspondence Address: LERNER, DAVID, LITTENBERG,;KRUMHOLZ & MENTLIK, 600 SOUTH AVENUE WEST, WESTFIELD, NJ, 07090, US

(73) Assignees: Centre National de la Recherche Scientifique(CNRS), Paris (FR); Universite de Luxembourg, Luxembourg (LU)

(21) Appl. No.: 12/224,692

(22) PCT Filed: Mar. 2, 2007
(86) PCT No.: PCT/EP2007/051994

§ 371 Date: Oct. 14, 2009

(30) Foreign Application Priority Data
Mar. 3, 2006 (FR) 0601906

(51) Int. Cl. A61K 31/085; C07C 39/12; C07C 43/20; A61K 31/05; A61P 25/00

(52) U.S. Cl. 514/721; 568/729; 568/660; 514/736

(57) ABSTRACT

The present invention relates to a compound of general formula (I) below in which R₁, R₂ and R₃ represent, independently of one another, a hydrogen atom or a C₁-C₆ alkyl group or a (C₁-C₆ alkyl)carbonyl group, R₄, R₅, R₆ and R₇ represent a hydrogen or a C₁-C₆ alkyl group, a C1-C6 alkoxy group or a (C₁-C₆ alkyl)carbonyloxy group, and n is an integer between 8 and 20, or its pharmaceutically acceptable addition salts, isomers, enantiomers and diastereoisomers, and also mixtures thereof. The invention also relates to a pharmaceutical composition comprising the compound and to the use thereof as a neurotrophic agent.

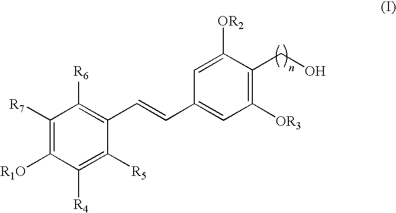

The present invention relates to a neurotrophic, neuroprotective and/or anti-inflammatory compound, in particular a resveratrol derivative carrying a long chain ω-alkanol of general formula (I) below:

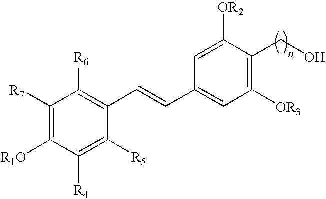

in which R₁, R₂ and R₃ represent, independently of one another, a hydrogen atom, a C₁-C₆ alkyl group or a (C₁-C₆ alkyl)carbonyl group, R₄, R₅, R₆ and R₇ represent a hydrogen atom, a C₁-C₆ alkoxy group, a C₁-C₆ alkyl group or a (C₁-C₆ alkyl)carbonyloxy group, and n is an integer between 8 and 20, preferably between 10 and 16.

The central nervous system (CNS) is comprised of two cellular populations which can be described as neural cells, namely neurons and all the other cell types, named glial cells. Astrocytes and oligodendrocytes represent the major types of glial cells in the adult. These cells are generated throughout fetal development and their production continues after birth and until adulthood in certain areas of the brain.

Neurons, astrocytes and oligodendrocytes are derived from a common precursor, a multipotent cell with theoretically unlimited proliferation capacities, which is localized in the neural tube. These precursors can be regarded as neural stem cells because they meet the criteria that define stem cells: self-renewal, quasi infinite proliferation capacity and the capacity to generate the cell types that constitute the tissue from which they arise.

Neurons, which are hyper-specialized cells, develop within a support and maintenance tissue, the glia. Central glia are those glial cells of the central nervous system and peripheral glia are those glial cells of the peripheral nervous system. More precisely, the central glia are comprised of astrocytes (astroglia), oligodendrocytes (oligodendroglia) and microgliacytes (microglia). The peripheral glia are comprised of Schwann cells, which are equivalent to the oligodendrocytes of the central glia.

Astrocytes are components of the blood-brain barrier (BBB). They intervene in the regulation of cerebral metabolism and serve as an interface between capillaries and neurons (nutritive role) via projections (pseudopods) which wind around the capillaries. They take part in neurotransmitter reuptake and also intervene in healing by producing glial filaments (components of the cytoskeleton).

Microgliacytes are glial cells, originating in the myeloid series, which invade the central nervous system during the embryonic period. Microglia are specialized in the elimination of macromolecules, the phagocytosis of cells in apoptosis or necrosis, the recognition and elimination of pathogenic elements, as well as in the regulation of the immune response.

Oligodendrocytes ensure the myelination of axons in the central nervous system. There is a diversity of progenitors at the origin of oligodendrocytes. These cells are generated in highly restricted ventricular zones along the neural tube. In the adult, oligodendrocytes are dispersed throughout the cerebral parenchyma, with predominance in the white matter fasciculi.

The myelin sheath, synthesized by oligodendrocytes, is a membrane protein complex wrapped around the axons. It has two functions: it acts as an electrical insulator and, more importantly, it increases the speed at which nerve impulses propagate. An attack on this sheath will cause the action potential to slow, or even to stop, thus disturbing the transmission of information in the nerve and causing neurological disorders. The loss or degeneration of myelin leads to the appearance of so-called demyelinating or dysmyelinating disorders.

The public health problems posed by neurodegenerative and demyelinating disorders such as Alzheimer's disease and multiple sclerosis are undeniable, not to mention their economic stakes. These diseases, which remain incurable, cause the slow and highly progressive death of nerve cells; for the most part, no effective treatments are available.

The worldwide prevalence of Alzheimer's disease is 12 million, a figure that will triple in 50 years given the increases in life expectancy in developed countries.

Current treatments are symptomatic only and there is a strong demand for new drugs that are highly effective and that can be administered by peripheral route. The symptomatic drugs for Alzheimer's disease are primarily anticholinesterases (Aricept®, Exelon®, Reminyl®). The newest drug on the market is Namenda®, which is an N-methyl-D-Aspartate (NMDA) receptor inhibitor.

Moreover, one of most widespread and most devastating demyelinating diseases is multiple sclerosis (MS), which is a progressive inflammatory disease of the nervous system characterized by degeneration of the myelin sheath, oligodendrocytes and neurons.

Multiple sclerosis affects approximately 2.5 million people worldwide, including a large number of young adults (between 20 and 40 years of age).

Treatments for multiple sclerosis are divided among four immunomodulator drugs (Rebif®, Avonex®, Betaseron® and Copaxone®). Current treatments take the inflammatory component into account relatively well. However, they prove to be inactive on the demyelinating component, the cause of the permanent and cumulative handicap. As opposed to what was formerly believed, it turns out that even at the beginning of the disease the oligodendrocytes maintain the capacity to manufacture new myelin (remyelination). In the chronic stage, on the other hand, this remyelination capacity appears completely lost. Thus it is known that in most patients, MS initially evolves in “relapsing/remitting” form and then in “progressive” form. It seems clear that oligodendrocytes and their precursors can survive the inflammatory phenomena of the first phase, but that in contrast their number and their effectiveness are reduced considerably during the chronic phase. Two therapeutic possibilities have been considered so far: transplantation of oligodendrocyte precursors or stimulation of myelination by chemical substances from surviving oligodendrocytes and endogenous oligodendrocyte precursors (endogenous remyelination).

With respect to transplantation, the use of oligodendrocyte precursors (relatively undifferentiated young cells), which are considered as having a greater potential for myelin synthesis and migration compared to adult oligodendrocytes, is foreseen in the prior art as capable of repairing a maximum number of demyelinated zones. The ideal source of oligodendrocytes is very young (embryonic) human nerve tissue, which raises ethical and practical problems. In addition, their migratory properties are unknown. Yet the problem is that, in most patients, there are multiple lesions and it is illusory to “transplant” each one individually. Furthermore, it is still not known if these oligodendrocytes are capable of migrating by themselves or if intervention by specific chemical factors is essential, not to mention that the longevity of these cells is unknown.

Moreover, in the prior art, various neurotrophic factors (L. Shen, A. Figurov & B. Luu, Journal of Molecular Medicine, 1997, vol. 75, 637-644) have been described which control neuron survival, growth and differentiation and are thus specific growth factors in the brain. They protect nerve cells against various attacks, in particular against substances released by activated microglial cells responsible for inflammation within the brain as well as for various disorders due to faulty operation of the nervous system.

However, these neuroprotective macromolecules cross the blood-brain barrier with difficulty. Thus the need for neurotrophic lipophilic molecules that can be administered by systemic route for the treatment of central nervous system diseases, and as a result the need to synthesize trophic molecules capable of inducing the differentiation of neural stem cells and other cellular precursors.

Within the framework of the present invention, the inventors looked into the possible actions of new compounds on so-called stem cells (M. Brehm, T. Zeus & B. E. Strauer, Herz, 2002, vol. 27, 611-620).

The mechanisms by which a neural stem cell gives rise to the three major types of CNS cells are still poorly understood. It is known, however, that this development proceeds by successive stages during which the developmental potential of the neural stem cell is gradually restricted. Thus, intermediate precursors with increasingly limited potentials are produced that lead to highly differentiated cells. Initially, stem cells are of embryonic origin. They are thus primarily present in fetuses and infants. They are able to multiply practically without limit. Under the action of these neurotrophic factors, they are transformed into mature and functional cells of various types (M. Y. Chang, H. Son, Y. S. Lee & S. H. Lee, Molecular and Cellular Neuroscience 2003, vol. 23N 3, 414-426). Quite recently, it was shown that stem cells are also present in organs in more developed stages, in particular in the brain (Ph. Taupin & F. H. Gage, Journal of Neuroscience Research, 2002, vol. 69, 745-749) and the spinal cord of adults. Thus, under the action of various growth factors, neural stem cells, i.e., stem cells present in the brain, can be transformed into neurons, astrocytes or oligodendrocytes, i.e., the principal types of nerve cells.

However, because of their molecular size and their physicochemical properties, these natural protein growth factors cannot cross the various biological barriers, in particular the blood-brain barrier. Thus they cannot penetrate into the brain in sufficient quantities to exert their beneficial actions. Moreover, they possess very poor bioavailability, thus limiting their effectiveness and their use.

Furthermore, when stem cells are used to treat degenerative neuropathies, they are introduced into the brain using a surgical procedure that is a risky invasive step (C. N. Svendsen & M. A. Caldwell, Progress in Brain Research, 2000, vol. 127, 13-34), as can be the case with oligodendrocyte precursors as well.

The present invention proposes an advantageous alternative to transplantation. Indeed, the inventors have discovered in a surprising and unexpected way that if an alkanol chain is grafted on a resveratrol core, molecules are obtained that cross the blood-brain barrier and that encourage endogenous remyelination by modulating the cellular specification of neural stem cells, i.e., that influence the choice for a neural stem cell to orient itself towards the neuronal or glial pathway (modulation of the neuron/glial cell ratio). In addition, these compounds are capable of mimicking the action of certain neurotrophic factors. These mimics are able to transform neural stem cells into differentiated nerve cells and, in particular, to preferentially induce the formation of neurons in situ in the brain. This preferential neuron formation is particularly advantageous for the treatment of Alzheimer's disease. Thus, the compounds according to the invention are particularly suited for use in the treatment of Alzheimer's disease.

More particularly, the compounds according to the invention encourage neuronal survival and neurite growth. The compounds according to the invention are also able to decrease the inflammatory component of diseases affecting the nervous system. They can thus, in particular, decrease the activation of microglia and/or astrocytes.

These compounds are also able to decrease reactive gliosis, i.e., glial scarring, more particularly by modulating the expression of certain compounds of the astrocyte cytoskeleton. The repression of the activation of microglial cells and the transformation of neural stem cells into mature oligodendrocyte cells are essential characteristics for the treatment of neurodegenerative or demyelinating/dysmyelinating type diseases such as, in particular, multiple sclerosis, Alzheimer's disease, Parkinson's disease, Creutzfeldt-Jakob disease, but also vascular dementia, amyotrophic lateral sclerosis, infantile spinal muscular atrophy and neuropathy related to cerebral vascular accidents.

The antioxidant capacity of resveratrol is due to the presence of hydroxyl groups and more particularly to that in position 4′:

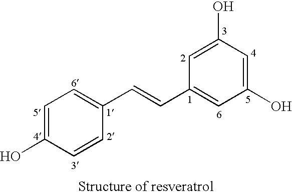

Hydroxyl radicals are highly reactive electrophilic species, and thus they easily react by substitution with aromatic compounds.

The hydroxyl radical preferentially will be added at the 3′ activated position on the aromatic ring via the electron donor effect of the OH group in position para 4′.

Resveratrol is a polyphenol present in wine, tea and various fruits. Over the past ten years a growing interest in this compound has been observed. This is partly connected to a study undertaken in the south of France revealing an inverse correlation between wine consumption and cardiovascular disease; this phenomenon is called the French Paradox [1]. In addition, resveratrol is found in the extract of the Polygonum cuspidatum plant, which has long been used in traditional Chinese medicine for its anti-inflammatory properties and in the treatment of certain cutaneous allergies.

Thus, the present invention relates to long-chain hydrocarbon alcohols substituted by a resveratrol core, called resveratrol fatty alcohols (RFAs), as well as analogs thereof, and in particular compounds of general formula (I) below:

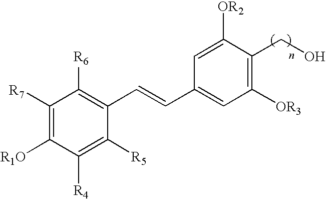

wherein R₁, R₂ and R₃ represent, independently of one another, a hydrogen atom, a C₁-C₆ alkyl group or a (C₁-C₆ alkyl)carbonyl group, R₄, R₅, R₆ and R₇ represent a hydrogen atom, a C₁-C₆ alkoxy group, a (C₁-C₆ alkyl)carbonyloxy group and n is an integer between 8 and 20, preferably between 10 and 16, or the pharmaceutically acceptable addition salts, isomers, enantiomers or diastereoisomers thereof, as well as mixtures thereof.

In the sense of the present invention, the term “pharmaceutically acceptable acid” means any non-toxic acid, including organic and inorganic acids. Such acids include acetic, benzenesulfonic, benzoic, citric, ethanesulfonic, fumaric, gluconic, glutamic, hydrobromic, hydrochloric, lactic, maleic, malic, mandelic, methanesulfonic, mucic, nitric, pamoic, pantothenic, phosphoric, succinic, sulfuric, tartaric and paratoluenesulfonic acid.

In the sense of the present invention, the term “C₁-C₆ alkyl group” means any linear or branched alkyl group with 1 to 6 carbon atoms, in particular a methyl, ethyl, n-propyl, isopropyl, n-butyl, isobutyl, sec-butyl, t-butyl, n-pentyl or n-hexyl group. Advantageously said group is a methyl group.

In the sense of the present invention, the term “C₁-C₆ alkoxy group” means any linear or branched alkoxy group with 1 to 6 carbon atoms, in particular a methoxy group.

In the sense of the present invention, the term “(C₁-C₆ alkyl)carbonyl group” means any

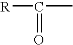

group wherein R represents a C₁-C₆ alkyl radical as defined above. Examples of (C₁-C₆ alkyl)carbonyl radicals include, but are not limited to, acetyl, propionyl, n-butyryl, sec-butyryl, t-butyryl, iso-propionyl groups and the like. In particular said group is an acetyl group.

In the sense of the present invention, the term “(C₁-C₆ alkyl)carbonyloxy group” means any

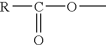

group wherein R represents a C₁-C₆ alkyl radical as defined above. In particular said group is an acetate group.

According to one embodiment of the invention, the compounds of general formula (I) correspond to those for which R₁, R₂ and R₃ represent a methyl (Me) group or a hydrogen atom.

According to another embodiment of the invention, the compounds of formula (I) correspond to those for which R₁, R₂ and R₃ represent a methyl group and R₄, R₅, R₆ and R₇ a hydrogen atom or a methoxy (OMe) group. Advantageously, R₅, R₆ and R₇ represent a hydrogen atom.

The side chain of the compound of formula (I) corresponds to a ω-alkanol wherein n is between 10 and 18.

The compounds of formula (I) wherein n is an integer equal to 10, 12, 14, 16 or 18 are particularly effective compounds within the framework of the present invention.

The compounds comprising a side chain of 10 or 12 carbon atoms are particularly advantageous.

The compounds still more particularly advantageous are:

RFA-12 of the formula below:

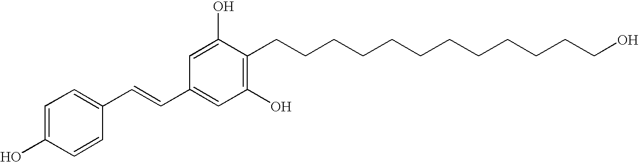

MRFA-12 of the formula below:

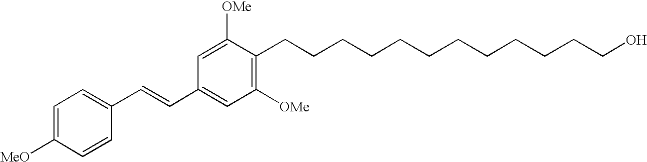

DMRFA-12 of the formula below:

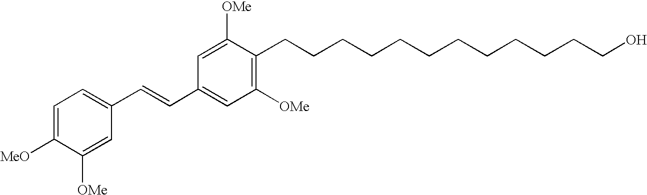

MRFA-10 of the formula below:

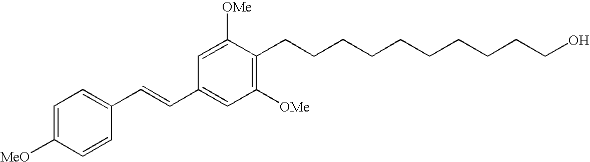

and DMRFA-10 of the formula below:

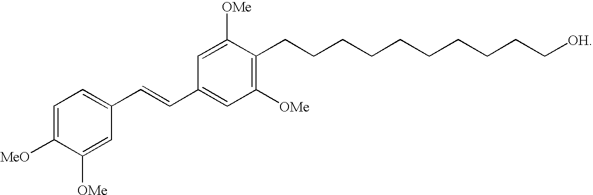

Advantageously, according to the present invention, the compound is RFA-12.

Thus, one object of the invention relates to a pharmaceutical composition comprising as active substance at least one long-chain hydrocarbon alcohol substituted by a resveratrol core, in particular an compound according to the invention of general formula (I) as previously defined, in combination with a pharmaceutically acceptable excipient.

Whatever the route of administration chosen, the advantageous inventive compositions are provided in a form favorable to the protection and the optimal assimilation of the active ingredient.

The compounds or compositions according to the invention can be administered in various ways and in various forms. Thus, said compounds or compositions can be administered systemically, by oral route, by inhalation, or by injection, such as, for example, by intravenous, intramuscular, subcutaneous, transdermal or intra-arterial route, etc., with intravenous, intramuscular, subcutaneous, oral and inhalation routes being advantageous.

For injections, the compounds are generally packaged in the form of liquid suspensions, which can be injected by means of syringes or perfusions, for example. In this respect, the compounds are generally dissolved in saline, physiological, isotonic or buffered solutions, etc., compatible with pharmaceutical use and known to those persons skilled in the art. Thus, the compositions can contain one or more agents or vehicles chosen among dispersants, solubilizers, stabilizers, preservatives, etc. Agents or vehicles usable in liquid and/or injectable formulations are notably methylcellulose, hydroxymethylcellulose, carboxymethylcellulose, polysorbate 80, mannitol, gelatin, lactose, vegetable oils, acacia, etc.

The compounds can also be administered in the form of gels, oils, tablets, suppositories, powders, gelatin capsules, capsules, aerosols, etc., possibly by means of galenic forms or by devices ensuring extended and/or delayed release. For this type of formulation, an agent such as cellulose, carbonate or starch is advantageously used.

The compounds according to the invention can in particular modulate the activation of microglial cells and astrocytes at concentrations of 10⁻⁵ M to 10⁻⁹ M, preferably 10⁻⁶ M to 10⁻⁸ M, even more preferentially 10⁻⁶ M. These compounds, at the same advantageous concentrations, induce the differentiation of neural stem cells into neurons as well as the survival of the neurons under these conditions.

The inventors having demonstrated the activity of the compounds according to the invention at the concentrations indicated above, it is understood that the flow rate and/or the dose injected can be adapted by those persons skilled in the art according to the patient, the pathology concerned, the administration route, etc. Moreover, repeated injections can be carried out, if necessary. In addition, for chronic treatments, delayed or extended systems can be advantageous.

The invention also relates to the pharmaceutical compounds and compositions according to the invention for the use of same as a drug.

The compounds and compositions according to the invention are used as a drug having a neurotrophic and/or neuroprotective and/or anti-inflammatory effect, and/or intended to prevent or to treat nervous system diseases or disorders that alter oligodendrocytes or other cells of the nervous system and/or diseases or disorders of nervous system inflammation, and/or degenerative neuropathies, and/or demyelinating or dysmyelinating disorders and/or cerebral vascular accidents or any other lesional attacks of the nervous system.

In particular, the compounds or pharmaceutical composition according to the invention prevents or treats multiple sclerosis, Alzheimer's disease, Parkinson's disease and Creutzfeldt-Jakob disease. Other diseases likely to be treated are vascular dementia, amyotrophic lateral sclerosis and infantile spinal muscular atrophy. Similarly, lesions derived from cerebral vascular accidents and all other lesional attacks of the nervous system are likely to be treated by the compounds and compositions according to the invention.

In the sense of the invention, the term “treatment” includes preventive and curative treatments that can be used alone or in combination with other agents or treatments. Moreover, the treatment may be used for chronic or acute disorders.

Another object of the invention relates to the use of the compounds according to the invention as a drug to modulate the cellular specification of neural stem cells, to favor the differentiation and then the survival of the neurons and glial cells in differentiation, to favor the differentiation of oligodendrocyte precursors cells into mature oligodendrocytes, and/or to decrease the activation of microglia and/or the activation of astrocytes and/or the reactive gliosis.

The invention thus relates to the use in vitro of at least one compound according to the invention to obtain differentiated neurons and/or glial cells from stem cells.

The compounds according to the invention can be prepared from commercial products by implementing a combination of chemical reactions known to persons skilled in the art, possibly guided by the method for preparing compounds of general formula (I) as defined below [2].

The compounds of general formula (I), wherein R₁, R₂ and R₃ represent a hydrogen atom, can be obtained notably by a preparation method comprising step (a) of reacting the compound of general formula (I), wherein R₁, R₂ and R₃ represent a C₁-C₆ alkyl group, with boron tribromide in dichloromethane at −78° C. [3]. Other solvents and other temperatures can be used.

The compounds of general formula (I), wherein R₁, R₂ and R₃ represent a C₁-C₆ alkyl group, can be obtained notably by a preparation method comprising step (b) of Wadsworth-Emmons coupling between the compound of general formula (II) below:

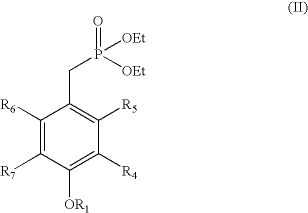

wherein R₁ represents a C₁-C₆ alkyl group and R₄, R₅, R₆ and R₇ are as defined above, and the compound of general formula (III) below [4]:

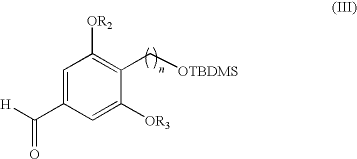

wherein R₂ and R₃ represent a C₁-C₆ alkyl group, TBDMS represents tert-butyl-dimethylsilyl and n is as defined above, advantageously in the presence of sodium methylate in dimethylformamide under reflux.

TBDMS is particularly desirable as a protective group because it can be cleaved in situ in the presence of 2 M HCl.

However, it is also possible to use another protective group for the OH group.

In particular, the compound of general formula (III) can be obtained by the preparation method comprising steps (c), (d) and (e) below:
(e) catalytic hydrogenation, advantageously with 5% palladium on carbon, of the compound of general formula (IV) below [5]:

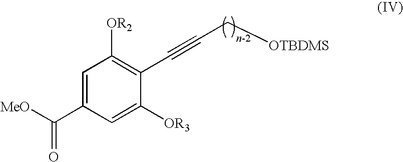

wherein R₂ and R₃ represent a C₁-C₆ alkyl group, and n is as defined above, to obtain the compound of general formula (V) below:

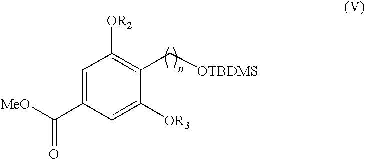

wherein R₂ and R₃ represent a C₁-C₆ alkyl group, and n is as defined above;
(d) reduction of the ester of formula (V) into the alcohol of general formula (VI) below:

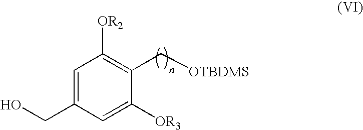

wherein R₂ and R₃ represent a C₁-C₆ alkyl group, and n is as defined above, advantageously using lithium aluminum hydride;
(c) oxidation of the alcohol of formula (VI) into the aldehyde of formula (III), advantageously using tetra-n-propylammonium perruthenate and N-methylmorpholine.

In particular, the compound of general formula (IV) can be obtained by the preparation method comprising step (f) of the Sonogashira coupling reaction between the compound of general formula (VII) below:

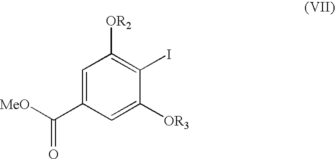

wherein R₂ and R₃ represent a C₁-C₆ alkyl group, and the compound of general formula (VIII) below [6]:

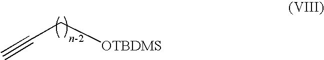

wherein n is as defined above, advantageously using PdCl₂(PPh₃)₂, in the presence of CuI in triethylamine under reflux.

Advantageously, within the framework of the method according to the invention, R₁, R₂ and R₃ represent a methyl group and R₄, R₅, R₆ and R₇ represent a hydrogen atom or a methoxy group.

Thus, RFA synthesis rests principally on two key steps. The first step is a Sonogashira coupling reaction between an aromatic iodide and the various chain lengths (n=10, 12, 14, 16, 18) in the form of true alkynes. The second step implements a Wadsworth-Emmons reaction between an aldehyde and the phosphonate formed in situ from the benzyl bromide obtained by bromination of the corresponding benzyl alcohol (diagram 1).

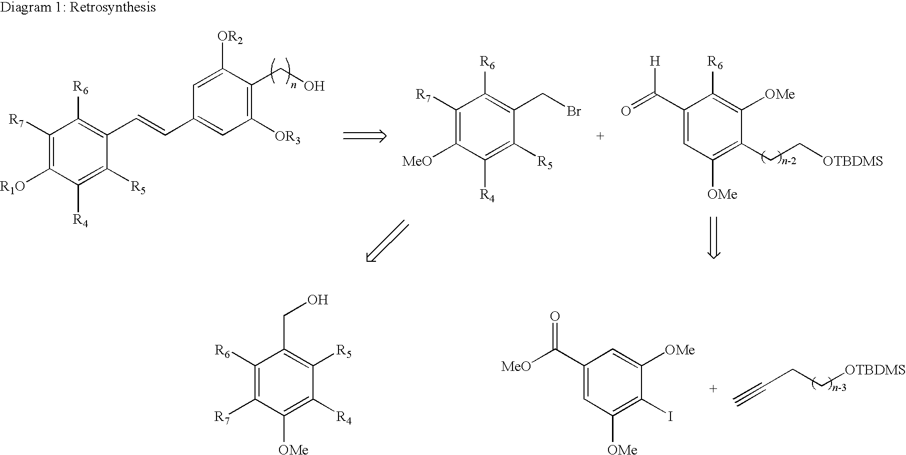

Synthesis of the Side Chain

In order to synthesize the various chain lengths necessary for Sonogashira coupling, the inventors followed the following reaction pathway (Diagram 2):

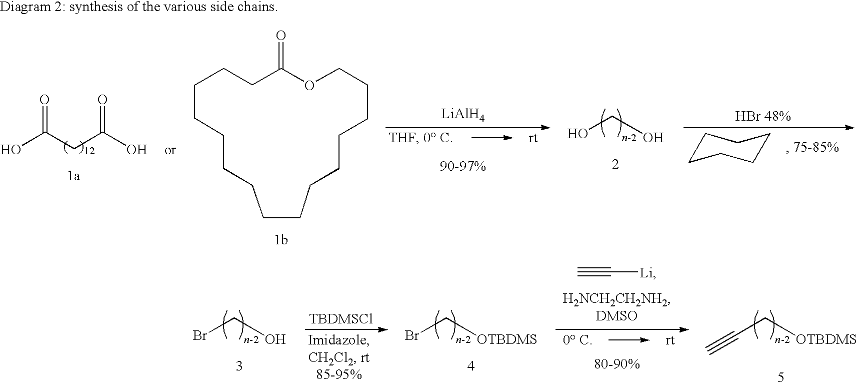

After reduction of diacid 1a or lactone 1b using lithium aluminum hydride, corresponding diols 2 are obtained. For the other chain lengths, commercial diols are used. The monobromination reaction is carried out in a heterogeneous mixture of hydrobromic acid and cyclohexane in order to obtain bromoalcohols 3 with good yields.

The inventors chose tert-butyl-dimethylsilyl as the protective group for the alcohol function because it is easily deprotected using TBAF. Finally, acetylene derivatives 5 are obtained by the action of lithium acetylide in DMSO on corresponding silylated bromoalcohols 4.

Synthesis of Aromatic Iodide

Methyl 4-iodo-3,5-dimethoxybenzoate 7 is obtained in two steps with a quite acceptable yield of 87%. First, the inventors treated 3,5-dihydroxybenzoic acid with a methanol solution of N-iodosuccinimide (NIS) in order to form iodine derivative 6. Then, the inventors methylated the hydroxyl functions to form desired compound 7 necessary for Sonogashira coupling.

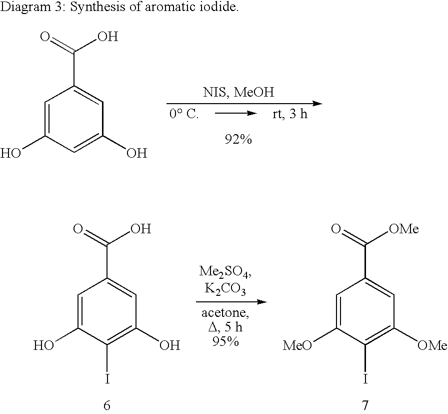

Sonogashira Coupling

This reaction is used to graft the various ω-hydroxylated chains 5 on the aromatic ring 7 (diagram 4).

Given that iodized derivatives generally have higher reactivity, the inventors decided to carry out the Sonogashira reaction with an iodine on benzyl ester 7 (diagram 3), although the latter is not commercially available.

The coupling reaction is thus performed according to the following reaction pathway:

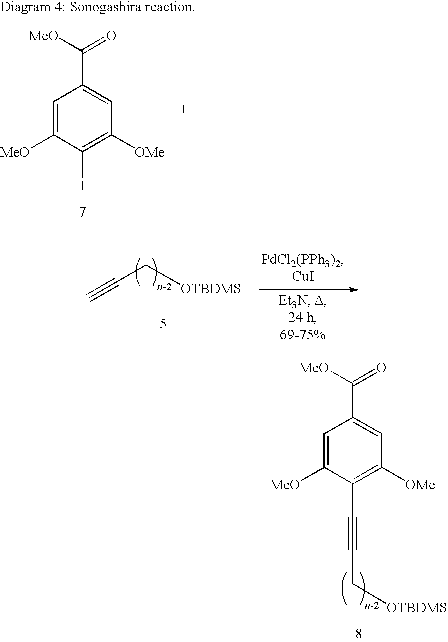

Now that the ω-alkanol chain is grafted, the following step is the synthesis of the aldehyde necessary for Wadsworth-Emmons coupling to form the trans C—C double bond between the two aromatic rings (diagram 5).

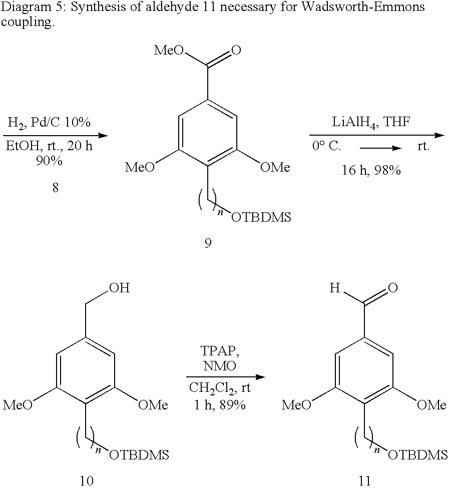

The compound with saturated chain 9 is obtained by catalytic hydrogenation with 5% palladium on carbon. In order to form aldehyde 11 for the Wadsworth-Emmons reaction, the inventors reduced ester 9 into alcohol 10 using lithium aluminum hydride. The inventors then oxidized the alcohol into aldehyde 11 using tetra-n-propylammonium perruthenate (TPAP) [(n-Pr₄N)RuO₄] and N-methylmorpholine.

Wadsworth-Emmons Coupling

The initial goal being to synthesize trans-resveratrol derivatives, the inventors chose a variant of the Wittig reaction which exclusively forms the double carbon-carbon bond with the trans configuration, namely Wadsworth-Emmons coupling (diagram 6).

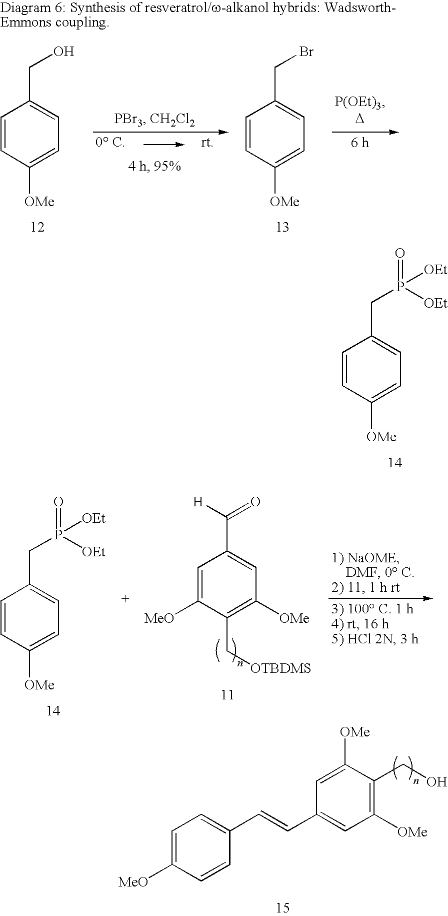

Generally, this reaction is based on coupling between an aldehyde and a phosphonate. Benzyl alcohol 12 is brominated using phosphorus tribromide (PBr₃) to form corresponding benzyl bromide 13, which in the presence of triethyl phosphite (POEt₃) under reflux forms desired phosphonate 14. Thus, the inventors reacted the phosphonate and the aldehyde previously formed in the presence of sodium methylate in dimethylformamide under reflux. During this reaction an intermediary is formed, a betaine, which by the elimination of triethyl phosphite oxide leads to the formation of the desired coupling product 15. The inventors thus formed various methoxylated RFAs which will also be tested.

RFAs 16 are finally obtained by the action of boron tribromide in dichloromethane at −78° C. (diagram 7).

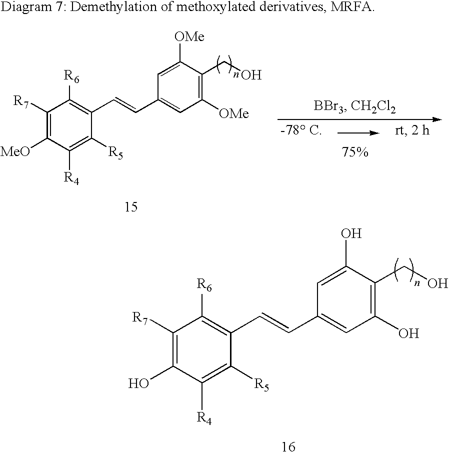

The following examples are given as illustrations and should not be regarded as limiting the present invention.

### EXAMPLES

### Example 1

### Preparation of 12-bromododecan-1-ol (3)

48% HBr in water (77 ml; 0.46 mol; 15 eq.) is added to a solution of 1,12-dodecan-ol (6.15 g; 30.4 mmol; 1 eq.) in cyclohexane (140 ml). The heterogeneous mixture is heated under reflux. After 6 h, the aqueous phase is extracted with ether (3×100 ml). The organic phases are combined, washed with a saturated solution of Na₂CO₃, dried on MgSO₄, filtered and evaporated. The crude reaction product is purified by silica gel chromatography (eluent: hexane-AcOEt: 6-4) to yield 7.06 g of a white solid.

Yield: 85%

Empirical formula: C₁₂H₂₅BrO

MW: 265.23

TLC: (hexane-AcOEt: 6-4) Rf=0.4

Compounds wherein n=8, 10, 14 and 16 were synthesized according to the same procedure as described above.

¹H NMR (300 MHz, CDCl₃) δ: 1.26 (s broad, 16H, H-3 to H-10); 1.56 (m, 2H, H-2); 1.85 (q, 2H, J=7.0 Hz, H-11); 3.40 (t, 2H, J=6.8 Hz, H-12); 3.63 (t, 2H, J=6.4 Hz, H-1).

¹³C NMR (75 MHz, CDCl₃) δ: 25.5 (C-3); 28.2 (C-11); 28.7 (C-9); 29.4 (C-4 to C-8); 32.8 (C-2); 33.8 (C-12); 63. (C-1).

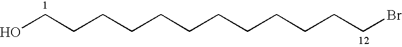

The ¹H NMR and ¹³C NMR spectra for the other chain lengths are identical with at each time ±4H at 1.26 ppm and ±2 C at 29.4 ppm.

### Example 2

### Preparation of (12-bromododecyloxy)(tert-butyl)dimethylsilane (4)

Tert-butyldimethylsilyl chloride (5.61 g; 37.3 mmol; 1.5 eq.) and imidazole (2.54 g; 37.3 mmol; 1.5 eq.) are added to a solution of 12-bromododecan-1-ol (6.6 g; 24.9 mmol; 1 eq.) in dichloromethane (60 ml) at room temperature. After 4 h, the reaction mixture is poured into a saturated solution of NH₄Cl (100 ml) and then extracted with ether (3×100 ml). The organic phase is washed with a saturated solution of NaCl, dried on MgSO₄, filtered then evaporated. The crude reaction product is purified by silica gel chromatography (eluent: hexane-AcOEt: 95-5) to yield 9.17 g of a colorless oil.

Yield: 99%

Empirical formula: C₁₈H₃₉BrOSi

MW: 379.49

TLC: (hexane-CH₂Cl₂: 8-2) Rf=0.5

Compounds wherein n=8, 10, 14 and 16 were synthesized according to the same procedure as described above.

¹H NMR (300 MHz, CDCl₃) δ: 0.04 (s, 6H, CH₃Si); 0.89 (s, 9H, CH₃C); 1.27 (s broad, 16H, H-3 to H-10); 1.39 to 1.53 (m, 2H, H-2); 1.85 (q, 2H, J=6.9 Hz, H-11); 3.40 (t, 2H, J=6.9 Hz, H-12); 3.59 (t, 2H, J=6.6 Hz, H-1).

¹³C NMR (75 MHz, CDCl₃) δ: −5.2 (CH₃Si); 18.4 (CSi); 26.0 (CH₃C); 28.2-29.6 (C-2 to C-10); 32.9 (C-11); 34.0 (C-12); 63.4 (C-1)

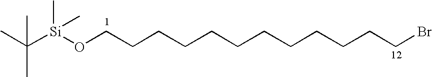

¹H NMR and ¹³C NMR spectra for the other chain lengths are identical with at each time ±4H at 1.27 ppm and ±2 C at 28.2-29.6 ppm.

### Example 3

### Preparation of tert-butyl(tetradec-13-ynyloxy)dimethylsilane (5)

A solution of (12-bromododecyloxy)(tert-butyl) dimethylsilane (9.2 g; 24.2 mmol; 1 eq.) in DMSO (5 ml) is added dropwise to a lithium acetylide solution (3.35 g; 36.4 mmol; 1.5 eq.) cooled to 0° C. in DMSO (15 ml). The reaction mixture is allowed to stir at room temperature for 16 h. The reaction mixture is poured into a saturated solution of KCl (100 ml) and then extracted with hexane (3×100 ml). The organic phase is washed, dried on MgSO₄, filtered and evaporated. The crude reaction product is purified by silica gel chromatography (eluent: hexane-CH₂Cl₂: 8-2) to yield 6.38 g of a colorless oil.

Yield: 81%

Empirical formula: C₂₀H₄₀OSi

MW: 324.62

TLC: (heptane-CH₂Cl₂: 8-2) Rf=0.4

Compounds wherein n=10, 12, 16 and 18 were synthesized by the same procedure as described above.

¹H NMR (300 MHz, CDCl₃) δ: 0.04 (s, 6H, CH₃Si); 0.90 (s, 9H, CH₃C); 1.27 (s broad, 16H, H-3 to H-10); 1.38 to 1.59 (m, 4H, H-2 and H-11); 1.93 (t, 1H, J=2.6 Hz, H-14); 2.18 (td, 2H, J=2.5 Hz and J=6.9 Hz, H-12); 3.59 (t, 2H, J=6.6 Hz, H-1).

¹³C NMR (75 MHz, CDCl₃) δ: −5.2 (CH₃Si); 18.4 (CSi and C-12); 26.0 (CH₃C); 25.8-29.9 (C-3 to C-11); 32.9 (C-2); 63.4 (C-1); 68.0 (C-14); 84.8 (C-13).

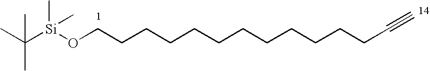

¹H NMR and ¹³C NMR spectra for the other chain lengths are identical with at each time+4H in addition at 1.27 ppm and ±2 C at 25.8-29.9 ppm.

### Example 4

### Preparation of 3,5-dihydroxy-4-iodobenzoic Acid (6)

A solution of N-iodosuccinimide (5 g; 22.2 mmol; 1.05 eq.) in methanol (15 ml) is added dropwise to a solution of 3,5-dihydroxybenzoic acid (3.26 g; 21.2 mmol; 1 eq.) cooled to 0° C. in methanol (13 ml). The reaction mixture is allowed to stir at room temperature for 3 h. The mixture is poured into cold water (13 ml) and then into a 5% aqueous solution of Na₂SO₃ (20 ml). The aqueous phase is extracted with ether (3×100 ml). The organic phase is washed with NaCl, dried on MgSO₄, filtered and evaporated to yield 5.37 g of a white solid.

Yield: 90%

Empirical formula: C₇H₅IO₄

MW: 280.02

TLC: (AcOEt) Rf=0.1

¹H NMR (300 MHz, CDCl₃) δ: 2.67 (s, 1H, H_{acid}); 4.91 (s, 2H, OH); 6.98 (s, 2H, H-3 and H-5).

¹³C NMR (75 MHz, CDCl₃) δ: 80.3 (C-1); 106.2 (C-3 and C-5); 131.8 (C-4); 158.0 (C-2 and C-6); 168.1 (C-7).

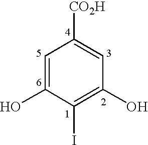

### Example 5

### Preparation of Methyl 4-iodo-3,5-dimethoxybenzoate (7)

Potassium carbonate (12 g; 85.8 mmol; 5.6 eq.) and dimethylsulfate (12.6 g; 99.6 mmol; 6.5 eq.) are added to a solution of 3,5-dihydroxy-4-iodobenzoic acid (4.3 g; 15.3 mmol; 1 eq.) in acetone (48 ml). The reaction mixture is heated under reflux for 4 h. Methanol (26 ml) is added and reflux is maintained for an additional hour. The mixture is poured into a saturated solution of NH₄Cl (100 ml) and then extracted with ether (3×100 ml). The organic phase is dried on MgSO₄, filtered and then evaporated. The crude reaction product is purified by silica gel chromatography (eluent: heptane-AcOEt: 6-4) to yield 4.66 g of a white solid.

Yield: 95%

Empirical formula: C₁₀H₁₁IO₄

MW: 322.10

TLC: (heptane-AcOEt: 7-3) Rf=0.4

¹H NMR (300 MHz, CDCl₃) δ: 3.93 (s, 3H, H-8); 3.94 (s, 6H, H-9 and H-10); 7.16 (s, 2H, H-2 and H-6).

¹³C NMR (75 MHz, CDCl₃) δ: 52.4 (OMe); 56.8 (OMe); 84.3 (C-1); 104.7 (C-3 and C-5); 131.8 (C-4); 159.5 (C-2 and C-6); 166.6 (C-7)

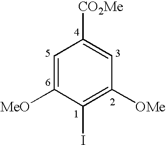

### Example 6

### Preparation of Methyl 4-(14-(tert-butyldimethylsilyloxy)tetradec-1-ynyl)-3,5-dimethoxybenzoate (8)

Palladium complex PdCl₂(PPh₃)₂ (200 mg; 0.28 mmol; 0.07 eq.) and copper iodide (55 mg; 0.28 mmol; 0.07 eq.) respectively are added to a solution of methyl 4-iodo-3,5-dimethoxybenzoate (1.3 g; 4.04 mmol; 1 eq.) in triethylamine (11 ml). The reaction mixture is allowed to stir at room temperature for 10 minutes, then tert-butyl(dodecan-13-ynyloxy)dimethylsilane (1.97 g; 6.05 mmol; 1.5 eq.) is added. After 48 h of heating under reflux, the mixture is poured into a saturated solution of NH₄Cl (100 ml) and then extracted with ethyl acetate (3×100 ml). The organic phase is washed with a saturated solution of NaCl, dried on MgSO₄, filtered and evaporated. The crude reaction product is adsorbed on silica (200 μm) and then purified by silica gel chromatography (eluent: heptane-AcOEt: 9-1) to yield 1.31 g of a yellow oil.

Yield: 63%

Empirical formula: C₃₀H₅₀O₅Si

MW: 518.80

TLC: (eluent: heptane-AcOEt: 8-2) Rf=0.4

Compounds wherein n=10, 12, 16 and 18 were synthesized according to the same procedure as described above.

¹H NMR (300 MHz, CDCl₃) δ: 0.04 (s, 6H, CH₃Si); 0.89 (s, 9H, CH₃C); 1.27 (s broad, 16H, H-5′ to H-13′); 1.47 to 1.68 (m, 2H, H-4′); 2.55 (t, 2H, J=7.0 Hz, H-3′); 3.59 (t, 2H, J=6.6 Hz, H-14′); 3.92 (s, 9H, OMe); 7.21 (s, 2H, H-2 and H-6).

¹³C NMR (75 MHz, CDCl₃) δ: −5.3 (CH₃Si); 18.4 (CSi); 20.3 (C-3′); 26.0 (CH₃C); 28.8 to 29.5 (C-4′ to C-12′); 32.9 (C-13′); 52.3 (OMe); 56.3 (OMe); 63.3 (C-14′); 72.2 (C-1′); 102.5 (C-2′); 104.6 (C-2 and C-6); 107.0 (C-4); 129.9 (C-1); 161.0 (C-3 and C-5); 166.7 (C-7).

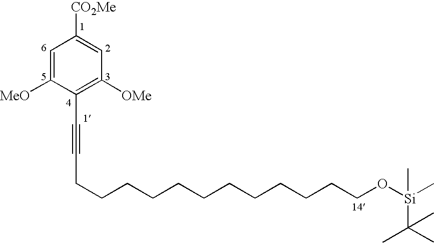

¹H NMR and ¹³C NMR spectra for the other chain lengths are identical with at each time ±4H in addition at 1.27 ppm and ±2 C at 28.8-29.5 ppm.

### Example 7

### Preparation of Methyl 4-(14-(tert-butyldimethylsilyloxy)tetradecyl)-3,5-dimethoxybenzoate (9)

10% palladium on carbon (256 mg; 0.24 mmol; 0.01 eq.) is added to a solution of methyl 4(14(tert-butyldimethylsilyloxy)tetradec-1-ynyl)-3,5-dimethoxybenzoate (1.28 g; 2.47 mmol; 1 eq.) in ethanol (5 ml). The reaction mixture is allowed to stir under a hydrogen atmosphere at room temperature. After 24 h, the mixture is filtered on celite. The crude reaction product is purified by silica gel chromatography (eluent: heptane-AcOEt: 7-3) to yield 1.23 g of a yellow oil.

Yield: 95%

Empirical formula: C₃₀H₅₄O₅Si

MM: 522.83

TLC: (eluent: heptane-AcOEt: 7-3) Rf=0.6

Compounds wherein n=10, 12, 16 and 18 were synthesized according to the same procedure as described above.

¹H NMR (300 MHz, CDCl₃) δ: 0.05 (s, 6H, CH₃Si); 0.89 (s, 9H, CH₃C); 1.25 (s broad, 20H, H-3′ to H-12′); 1.43 to 1.53 (m, 4H, H-2′ and H-13′); 2.65 (t, 2H, J=7.2 Hz, H-1′); 3.59 (t, 2H, J=6.6 Hz, H-14′); 3.85 (s, 6H, OMe); 3.91 (s, 3H, OMe); 7.22 (s, 2H, H-2 and H-6).

¹³C NMR (75 MHz, CDCl₃) δ: −5.2 (CH₃Si); 18.4 (CSi); 23.2 (C-1′); 25.9 (CH₃C); 28.9 to 29.8 (C-2′ to C-12′); 32.9 (C-13′); 52.1 (OMe); 55.8 (OMe); 63.4 (C-14′); 104.9 (C-2 and C-6); 125.4 (C-4); 128.4 (C-1); 158.0 (C-3 and C-5); 167.7 (C-7).

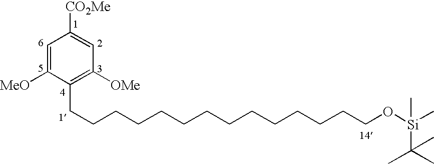

¹H NMR and ¹³C NMR spectra for the other chain lengths are identical with at each time ±4H in addition at 1.25 ppm and ±2 C at 28.9-29.8 ppm.

### Example 8

### Preparation of (4-(14-(tert-butyldimethylsilyloxy)tetradecyl)-3,5-dimethoxyphenyl)methanol (10)

Lithium aluminum hydride (87 mg; 2.3 mmol; 1 eq.) is added in small amounts to a solution of methyl 4(14(tert-butyldimethylsilyloxy)tetradecyl)-3,5-dimethoxybenzoate (1.2 g; 2.3 mmol; 1 eq.) in THF (9 ml) cooled to 0° C. The reaction mixture is allowed to stir at room temperature. After 2.5 h, the mixture is poured into a 10% aqueous solution of sodium tartrate (100 ml) and then extracted with ether (3×100 ml). The organic phase is washed, dried on MgSO₄, filtered and evaporated. The crude reaction product is purified by silica gel chromatography (eluent: heptane-AcOEt: 7-3) to yield 1.05 g of a colorless oil.

Yield: 92%

Empirical formula: C₂₉H₅₄O₄Si

MW: 494.82

TLC: (eluent: heptane-AcOEt: 7-3) Rf=0.3

Compounds wherein n=10, 12, 16 and 18 were synthesized according to the same procedure as described above.

¹H NMR (300 MHz, CDCl₃) δ: 0.05 (s, 6H, CH₃Si); 0.89 (s, 9H, CH₃C); 1.26 (s broad, 20H, H-3′ to H-12′); 1.29 to 1.52 (m, 4H, H-2′ and H-13′); 2.60 (t, 2H, J=7.2 Hz, H-1′); 3.59 (t, 2H, J=6.6 Hz, H-14′); 3.81 (s, 6H, OMe); 4.65 (s, 2H, H-7); 5.30 (s, OH); 6.55 (s, 2H, H-2 and H-6).

¹³C NMR (75 MHz, CDCl₃) δ: −5.2 (CH₃Si); 18.4 (CSi); 22.8 (C-1′); 26.0 (CH₃C); 25.8 to 29.8 (C-2′ to C-12′); 32.9 (C-13′); 55.7 (OMe); 63.4 (C-14′); 65.9 (C-7); 102.4 (C-2 and C-6); 119.0 (C-4); 139.5 (C-1); 158.4 (C-3 and C-5).

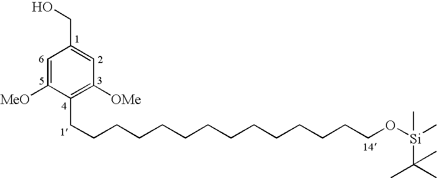

¹H NMR and ¹³C NMR spectra for the other chain lengths are identical with at each time+4H in addition at 1.26 ppm and ±2 C at 25.8-29.8 ppm.

### Example 9

### Preparation of 4-(14-(tert-butyldimethylsilyloxy)tetradecyl)-3,5-dimethoxybenzaldehyde (11)

N-methylmorpholine (422 mg; 3.12 mmol; 1.5 eq.) is added to a solution of (4(14(tert-butyldimethylsilyloxy)tetradecyl)-3,5-dimethoxyphenyl)methanol (1.03 g; 2.08 mmol; 1 eq.) in dichloromethane (5 ml) in the presence of 4 Å molecular sieves (500 mg/mmol). Tetra-n-propylammonium perruthenate (37 mg; 0.10 mmol; 0.05 eq.) is added in small amounts to the mixture cooled to 0° C. The reaction mixture is allowed to stir at room temperature for 2 h and then is filtered on celite. The crude reaction product is purified by silica gel chromatography (eluent: heptane-AcOEt: 7-3) to yield 855 mg of a colorless oil.

Yield: 85%

Empirical formula: C₂₉H₅₂O₄Si

MW: 492.81

TLC: (eluent: heptane-AcOEt: 6-4) Rf=0.6

Compounds wherein n=10, 12, 16 and 18 were synthesized according to the same procedure as described above.

¹H NMR (300 MHz, CDCl₃) δ: 0.05 (s, 6H, CH₃Si); 0.89 (s, 9H, CH₃C); 1.25 (s broad, 20H, H-3′ to H-12′); 1.29 to 1.52 (m, 4H, H-2′ and H-13′); 2.67 (t, 2H, J=7.2 Hz, H-1′); 3.59 (t, 2H, J=6.6 Hz, H-14′); 3.88 (s, 6H, OMe); 7.05 (s, 2H, H-2 and H-6); 9.90 (s, 1H, H-7).

¹³C NMR (75 MHz, CDCl₃) δ: −5.2 (CH₃Si); 18.4 (CSi); 23.5 (C-1′); 26.0 (CH₃C); 25.8 to 31.6 (C-2′ to C-12′); 32.9 (C-13′); 55.8 (OMe); 63.3 (C-14′); 104.9 (C-2 and C-6); 127.5 (C-4); 135.1 (C-1); 158.6 (C-3 and C-5); 191.9 (C-7).

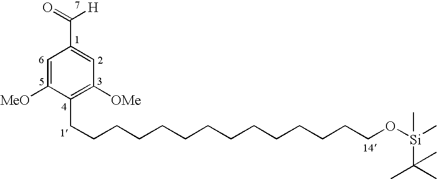

¹H NMR and ¹³C NMR spectra for the other chain lengths are identical with at each time+4H in addition at 1.25 ppm and ±2 C at 25.8-31.6 ppm.

### Example 10

### Preparation of 4-methoxybenzylic Bromide (13)

Phosphorus tribromide (6.2 g; 25.0 mmol; 1.15 eq.) is added to a 4-methoxybenzylic alcohol solution (3 g; 21.7 mmol; 1 eq.), cooled to 0° C., in dichloromethane (35 ml). After 5 h at room temperature, the reaction mixture is poured into ice water (100 ml) and then is extracted with ether (3×100 ml). The organic phase is washed, dried on MgSO₄, filtered and evaporated to obtain 4.1 g of a colorless oil.

Yield: 94%

Empirical formula: C₈H₉BrO

MW: 201.06

TLC: (eluent: heptane-AcOEt: 7-3) Rf=0.6

¹H NMR (300 MHz, CDCl₃) δ: 3.81 (s, 3H, OMe); 4.51 (s, 2H, CH₂Ph); 6.87 (d, 2H, J=8.8 Hz, H-3 and H-5); 7.32 (d, 2H, J=8.8 Hz, H-2 and H-6).

¹³C NMR (75 MHz, CDCl₃) δ: 34.0 (CH₂Ph); 55.3 (OMe); 114.2 (C-3 and C-5); 130.0 (C-1); 130.5 (C-2 and C-6); 159.7 (C-4).

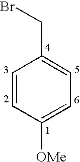

### Example 11

### Preparation of (E)-14-(4-(4-methoxystyryl)-2,6-dimethoxyphenyl)tetradecan-1-ol (II)

Triethylphosphite (565 mg; 3.44 mmol; 2 eq.) and 4-methoxybenzylic bromide (478 mg; 2.38 mmol; 1.4 eq.) are heated to 130° C. After 6 h, the reaction mixture is returned to room temperature and then a 5.4 M solution of sodium methylate (540 μl; 2.92 mmol; 1.7 eq.), dimethylformamide (2 ml) and a solution of 4-(14-(tert-butyldimethylsilyloxy)tetradecyl)-3,5-dimethoxybenzaldehyde (845 mg; 1.72 mmol; 1 eq.) in dimethylformamide (2 ml) are added respectively. The reaction mixture is allowed to stir at room temperature for 1 h and then is heated at 100° C. for an additional hour. The mixture is finally allowed to stir at room temperature overnight. A 2 M solution of HCl (6 ml) is added to the reaction mixture. After 3 h, the mixture is poured into a saturated KCl solution (100 ml) and then extracted with ether (3×100 ml). The organic phase is washed, dried on MgSO₄, filtered and evaporated. The crude reaction product is purified by silica gel chromatography (eluent: heptane-AcOEt: 7-3) to yield 568 mg of a white solid.

Yield: 69%

Empirical formula: C₃₁H₄₆O₄

MW: 482.69

TLC: (eluent: heptane-AcOEt: 7-3) Rf=0.2

Compounds wherein n=10, 12, 16 and 18 were synthesized according to the same procedure as described above.

¹H NMR (300 MHz, CDCl₃) δ: 1.26 (s broad, 20H, H-3″ to H-12″); 1.30 to 1.59 (m, 4H, H-2″ and H-13″); 2.61 (m, 2H, H-1″); 3.64 (t, 2H, J=6.6 Hz, H-14″); 3.83 (s, 3H, OMe); 3.86 (s, 6H, OMe); 6.67 (s, 2H, H-2 and H-6); 6.90 (d, 2H, J=8.8 Hz, H-3′ and H-5′); 6.98 (d, 1H, J=16.1 Hz, H-8); 7.02 (d, 1H, J=16.1 Hz, H-7); 7.45 (d, 2H, J=8.8 Hz, H-2′ and H-6′).

¹³C NMR (75 MHz, CDCl₃) δ: 23.0 (C-1″); 25.7 to 29.8 (C-2″ to C-11″); 32.8 (C-13″); 55.3 (OMe); 55.7 (OMe); 63.1 (C-14″); 101.9 (C-2 and C-6); 114.1 (C-3′ and C-5′); 119.4 (C-4); 127.2 (C-8); 127.4 (C-7); 127.6 (C-2′ and C-6′); 130.0 (C-1′); 136 (C-1); 158.4 (C-3 and C-5); 159.2 (C-4′).

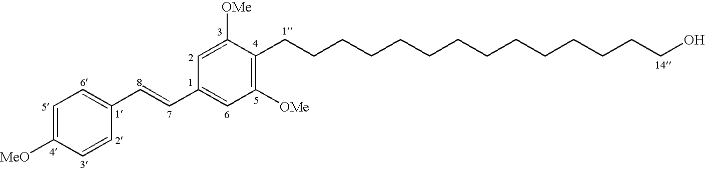

¹H NMR and ¹³C NMR spectra for the other chain lengths are identical with at each time ±4H in addition at 1.26 ppm and ±2 C at 29.3-29.8 ppm.

### Example 12

### Preparation of (E)-5-(4-hydroxystyryl)-2-(14-hydroxytetradecyl)benzene-1,3-diol (I)

Boron tribromide (230 μl; 2.42 mmol; 15 eq.) is added to a solution of (E)-14-(4-(4-methoxystyryl)-2,6-dimethoxyphenyl)tetradecan-1-ol (78 mg; 0.16 mmol; 1 eq.) in dichloromethane (5 ml) at −78° C. The reaction mixture is allowed to stir at room temperature. After 2 h, water (15 ml) is added at −78° C. then water is again added to the mixture (50 ml). The aqueous phase is extracted with AcOEt (3×50 ml). The organic phases are combined, washed with a saturated NaCl solution, dried on MgSO₄, filtered and evaporated. The crude reaction product is purified by silica gel chromatography (eluent: heptane-AcOEt: 45-55) to yield 53 mg of a white solid.

Yield: 75%

Empirical formula: C₂₈H₄₀O₄

MW: 440.61

TLC: (eluent: heptane-AcOEt: 4-6) Rf=0.3

Compounds wherein n=10, 12, 16 and 18 were synthesized by the same procedure as described above.

¹H NMR (300 MHz, CDCl₃) δ: 1.28 (s broad, 20H, H-3″ to H-12″); 1.40 to 1.52 (m, 4H, H-2″ and H-13″); 2.57 (m, 2H, H-1″); 3.53 (t, 2H, J=6.6 Hz, H-14″); 6.45 (s, 2H, H-2 and H-6); 6.74 (d, 1H, J=15.9 Hz, H-8); 6.75 (d, 2H, J=8.6 Hz, H-3′ and H-5′); 6.89 (d, 1H, J=15.9 Hz, H-7); 7.32 (d, 2H, J=8.6 Hz, H-2′ and H-6′).

¹³C NMR (75 MHz, CDCl₃) δ: 22.9 (C-1″); 25.7 to 29.8 (C-2″ to C-12″); 29.7 (C-13″); 64.3 (C-14″); 104.6 (C-2 and C-6); 115.4 (C-4); 115.9 (C-3′ and C-5′); 126.3 (C-8); 127.1 (C-7); 128.1 (C-2′ and C-6′); 128.6 (C-1′); 135.7 (C-1); 156.7 (C-3 and C-5); 156.8 (C-4′).

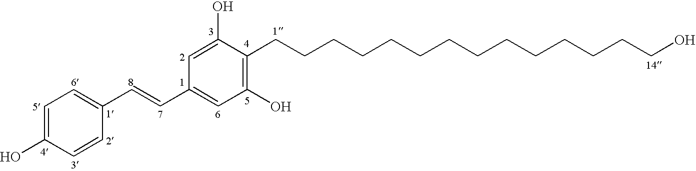

¹H NMR and ¹³C NMR spectra for the other chain lengths are identical with at each time+4H in addition at 1.28 ppm and ±2 C at 25.7-29.8 ppm.

### Example 13

### Physicochemical Study

The DPPH [(2,2′-di(4-tert-octylphenyl)1-picrylhydrazyl] test was used to determine the antioxidant capacity of our compounds [9]. DPPH is a stabilized radical that absorbs at 517 nm (violet color). When it is placed in the presence of an antioxidant molecule, the latter will react with the radical. Thus, a decrease in absorbance can be observed. Antioxidant capacity thus results in a decrease in absorbance at 517 nm.

a) Implementation of the DPPH Test

On a 96-well ELISA plate, add:
100 μl of the various solutions at the following concentrations: 10 mM, 5 mM, 2.5 mM, 1 mM, 0.5 mM, 0.1 mM, 0.01 mM and 0.001 mM;
100 μl of a 400 μM solution of DPPH in ethanol.

After incubating for 45 minutes, 2 hours and 3 hours, the result is given by measuring optical density at 550 nm using a spectrophotometer.

The result is expressed as IC₅₀, which is the concentration at which 50% of the radicals are reduced by the antioxidant molecule.

The inventors tested resveratrol, as a positive control, as well as RFA-10, RFA-12 and RFA-16.

Resveratrol has an IC₅₀ of 10 μM. Our hybrid molecules have an IC₅₀ of roughly 2.5 μM after 3 hours of incubation. It can be noted that antioxidant capacity is independent of the length of the C-hydroxylated chain. However, with an IC₅₀ of roughly 2.5 mM, our hybrid molecules maintain good antioxidant capacity.

The methoxylated derivatives were also tested. These compounds have an antioxidant capacity but at the concentrations tested the IC₅₀ is not reached.

b) Principle of the ABTS Test

In light of the results obtained with the DPPH test, the use of the ABTS ([2,2′-azino-bis-(3-ethylbenzthiazoline-6-sulfonic acid)]) test was considered [10], [11], [12]. The ABTS test is more sensitive insofar as it uses less encumbered hydroxyl radicals. In addition, it better reveals the antioxidant activity of compounds given that in the brain the prevalent radical is the hydroxyl radical.

The ABTS molecule reacts with the hydroxyl (OH) radicals generated in the medium by the Fenton reaction to form a cation radical, ABTS⁺ (green color), which is absorbent at 405 nm. Thus, when it is placed in the presence of an antioxidant molecule, one capable of reducing hydroxyl radicals, a reduction in absorbance at 405 nm can be observed.

On a 96-well ELISA plate, add:
180 μl of a 50/50 water/ethanol mixture;
30 μl of a 1 mM aqueous ABTS solution;
30 μl of a 1 mM aqueous FeSO₄ solution;
30 μl of the product to be tested at the following concentrations: 10 mM, 5 mM, 2.5 mM, 1 mM, 0.5 mM, 0.1 mM, 0.01 mM and 0.001 mM;
30 μl of a 100 mM aqueous H₂O₂ solution.

After incubating for 45 minutes, 1 hour and 2 hours, the result is given by measuring optical density at 405 nm using a spectrophotometer.

Since chain length does not determine antioxidant capacity, it was decided to perform this test with only one chain length. In order to maximally reduce solubility problems, RFA-10 was tested. Resveratrol was again used as a positive control.

The IC₅₀ values are those obtained after a 2 hours of incubation. Resveratrol has an IC₅₀ of 30 μM and RFA-10 an IC₅₀ of 45 μM.

In spite of resveratrol's slightly higher antioxidant capacity, our compounds exhibit a significant antioxidant potential.

### Example 14

### Biological Study

Inhibition of microglia activation by RFAs and MRFAs (resveratrol fatty alcohols and methoxylated resveratrol fatty alcohols). Production of neurons by neural stem cells.

In the brain, the immune system is represented by a population of monocyte-macrophage cells, microglial cells. Thus, the various tests used will evaluate the capacity of our compounds to modulate microglia activation as well their anti-inflammatory activity.

When the microglia is activated following various stimuli (lipopolysaccharides, interferon-gamma, etc.), a certain number of cytokines, such as TNF-α, or inducible enzymes, NOS-II and COX-II, are synthesized.

Thus, the level of nitric oxide (NO) and tumor necrosis factor-α (TNF-α) are indicators of microglial activation.

The experiments carried out thus relate to the capacity of RFA molecules (MRFA and DMRFA) to inhibit, in activated microglia, the release of nitrites and TNF-α.

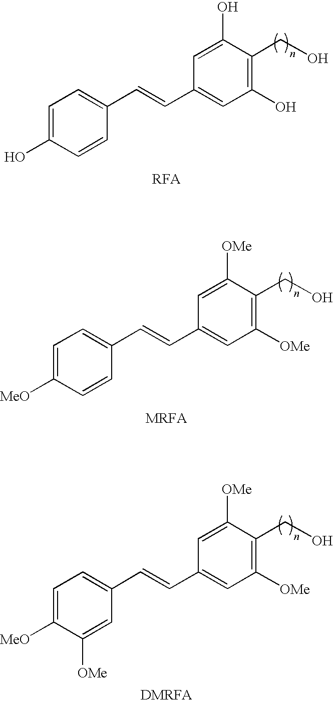

a) Measurements of Nitrite Release

The activity of NO-synthase type II (NOS II) represents a microglial activity parameter often analyzed in the literature. This enzyme is responsible for the synthesis of nitric oxide radicals under conditions of inflammatory activation. Microglia at rest only express levels at the immunoblotting detection limit. Activation of 24 to 48 hours leads to a strong increase in this expression. The product of this enzyme, NO, quickly breaks down in culture to form nitrites. A colorimetric assay (Griess method) shows that NO₂⁻ levels follow the same trend.

In the experiments performed, NO₂⁻ concentrations were measured after 24 hours and 48 hours of activation in microglial cell cultures treated with 0.01 μg/ml of LPS, in the presence of products RFA-12, MRFA-10, MRFA-12, MRFA-14, MRFA-16, MRFA-18, DMRFA-10, DMRFA-12, DMRFA-14, DMRFA-16, DMRFA-18, M-resveratrol, DM-resveratrol and resveratrol at concentrations of 5×10⁻⁶ M, 10⁻⁶ M and 10⁻⁷ M.

The results obtained for methoxylated and dimethoxylated derivatives, coming from three independent experiments, show that MRFA-10, DMRFA-10 and resveratrol induce in cells a 20% decrease in nitrite production compared to the control values.

In the RFA series, RFA-12 induces in cells a 50% decrease in nitrite levels at a concentration of 5×10⁻⁶ M.

These results show the importance of chain length as well as the presence of hydroxyl groups. Indeed, the best activity is observed for RFA-12, which is more active than resveratrol itself.

b) Measurements of TNF-α Release

The expression of TNF-α represents a microglial activation parameter often analyzed in the literature. Microglia at rest do not express this cytokine. A 24-hour activation by LPS leads to a strong increase in expression, detectable by ELISA.

In the experiments performed, TNF-α concentrations were measured after 24 hours of activation in microglial cell cultures treated with 0.01 μg/ml of LPS in the presence of MRFA-10, MRFA-12, MRFA-14, MRFA-16, MRFA-18, DMRFA-10, DMRFA-12, DMRFA-14, DMRFA-16, DMRFA-18, M-resveratrol, DM-resveratrol and resveratrol at concentrations of 5×10⁻⁶ M, 10⁻⁶ M and 10⁻⁷ M.

The results obtained for the MRFA series show that MRFA-10, MRFA-12 and MRFA-14, at a concentration of 5×10⁻⁶ M, lead in cells to a 20-40% reduction in TNF-α production compared to control values. The activity of these products is concentration dependant since it decreases with concentration. The methoxylated homologue of resveratrol, M-resveratrol, led to only a 20% decrease in TNF-α level.

Concerning the dimethoxylated derivative series (DMRFA), DMRFA-10 and DMRFA-12 induce at 5×10⁻⁶ M a 40% decrease in TNF-α compared to the control. A 50% decrease is observed at the same concentration for DM-resveratrol.

Finally, RFA-12 at a concentration of 5×10⁻⁶ M induces a 40% decrease in TNF-α compared to control values. Resveratrol induced a decrease of only 30% under the same conditions.

These preliminary results on TNF-α production show that, generally, the hybrid compounds according to the invention (RFA, MRFA and DMRFA) exhibit better activity than their resveratrol homologues. The importance of the presence of the ω-hydroxylated chain thus can be demonstrated, as well as its length. Indeed, the compounds carrying a 10- or 12-carbon-atom side chain prove to have the best activity.

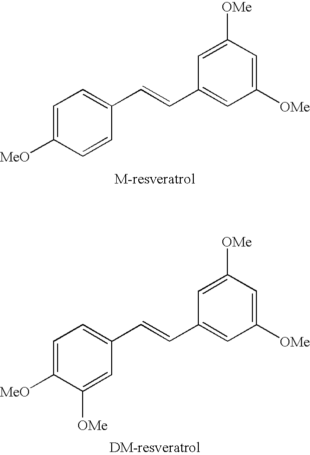

### Effect of RFA-12 and its Analogues on Neural Stem Cell Proliferation and Differentiation.

The majority of central nervous system (CNS) disorders are caused by degeneration of or attack on various cell types such as neurons or glial cells. Studies on animal models suggest that it may be possible to remediate this damage either by transplanting neural stem cells or by activating stem cells present within the central nervous system. These neural stem cells are capable of generating neurons, oligodendrocytes and astrocytes.

An interesting therapeutic approach is the development of small molecules such as RFA to control or induce the endogenous differentiation of neural stem cells into various nerve cells.

### Experimental Protocol

Neurospheres are obtained from mouse embryo telencephalon cells cultivated in a defined medium containing 20 ng/ml EGF. After dissociation, the spheres proliferate for 3 days in the presence of EGF (20 ng/ml) during the so-called proliferation phase.

The spheres are then collected and deposited on an poly-ornithine support in a defined medium in the presence of 2 ng/ml EGF. Under these conditions, the spheres adhere to the support and differentiate. The compounds to be tested, RFA-12 and its analogues, dissolved beforehand in ethanol, are added immediately after the deposit of the neurospheres at various concentrations ranging from 10⁻⁵ M to 10⁻⁹ M. The control cultures are treated with a negative control (ethanol) and a positive control (retinoic acid).

After 3 days of growth (differentiation phase) the cells are fixed and the various cell types are revealed by immunocytochemistry. To screen our compounds we performed a double labeling, namely for neurons and for astrocytes.

The primary antibodies used are:
anti-MAP2ab (microtubule associated protein) antibody, a marker for young neurons (mitotic);
TUJ1 (anti beta (III) tubulin) antibody, a marker for neurons (post-mitotic);
anti-GFAP (glial fibrillary acidic protein) antibody, a marker for astrocytes.
Anti-MAP2ab and TUJ1 antibodies are revealed by secondary antibodies coupled to a cyanine fluorochrome, Cy-3 (red). For the detection of astrocytes, a secondary antibody coupled to a fluorochrome derived from rhodamine, Alexa488 (green), was used.

In order to be able to quantify the results, the nuclei are marked by an intercalating agent, TO-PRO (blue). The cultures are then observed under a confocal microscope.

### Results

1. RFA-12 induces the differentiation of neural stem cells into neurons. The effect of RFA-12 on the differentiation of neural stem cells into neurons or glial cells was tested at concentrations of 10⁻⁵ M to 10⁻⁹ M. At a concentration of 10⁻⁵ M, it can be noted that RFA-12 does not induce differentiation due to toxicity at this concentration. In parallel, at 10⁻⁹ M RFA-12 exhibits only weak activity. However, if the neurospheres are treated with RFA-12 at a concentration of 10⁻⁶ M to 10⁻⁸ M, an increase in the number of neurons compared to the control (untreated cells) is observed. At concentrations of 10⁻⁶ M, 10⁻⁷ M and 10⁻⁸ M we can observe a 120%, 90% and 80% increase, respectively, in the neuron population compared to the control fixed at 100%. These results show that the effect of RFA-12 on the differentiation of neural stem cells into neurons is dose-dependant with a maximum effect at a concentration of 10⁻⁶ M.
2. In order to see whether RFA and its analogues also act on neural stem cell proliferation, these cells are dissociated and then cultured in the presence of RFA (10⁻⁶ M and 10⁻⁸ M) for 5 days, and then the number of living cells and their size and morphology are recorded.
The results show that RFA-12 at a concentration of 10⁻⁶ M inhibits the proliferation of neural stem cells.

### CONCLUSION

RFA-12, at a concentration of 10⁻⁶ M, induces the differentiation of neural stem cells into neurons while inhibiting the proliferation of these cells.

### REFERENCES

[1] S. Renaud and M. de Lorgeril, Lancet, 1992, 339, 1523-1526
[2] M. Wang, Y. Jin and C.-T. Ho, J. Agric. Food Chem., 1999, 47, 3974-3977
[3] G. R. Pettit, M. P. Grealish, M. K. Jung, E. Hamel, R. K. Pettit, J.-C. Chapuis and J. M. Schmidt, J. Med. Chem., 2002, 45, 2534-2542.
[4] W. P. Griffith and S. V. Ley, Aldrichimica Acta, 1990, 23, 13-19
[5] Y. Kondo, S. Kojima and T. Sakamoto, J. Org. Chem., 1997, 62, 6507-6511
[6] S. Hofman, Synthesis, 1998, 179-189
[7] WO 01/32633, 2001
[8] WO 03/031381, 2003
[9] P. Lorenz, S. Roychowdhury, M. Engelmann, G. Wolf, T. F. W. Horn, Nitric Oxide, 2003, 9, 64-76
[10] B. Poeggeler, S. Thuermann, A. Dose, M. Schoenke, S. Burkhardt and R. Hardeland, J. Pineal Res., 2002, 33, 20-30
[11] Z. Matuszak, K. J. Reska and C. F. Chignell, Free Radical Biology & Medicine, 1997, 23, 367-372.
[12] R. Reiter, L. Tang, J. J. Garcia and A. Munoz-Hoyos, Life Sciences, 1997, 60, 2255-2271.

1. A compound of general formula (I) below: 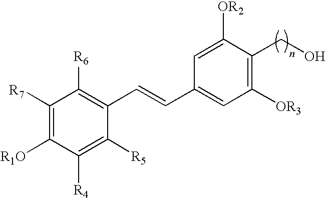
wherein
R₁, R₂ and R₃ represent, independently of one another, a hydrogen atom, a C₁-C₆ alkyl group or a (C₁-C₆ alkyl)carbonyl group, R₄, R₅, R₆ and R₇ represent a hydrogen atom, a C₁-C₆ alkyl group, a C₁-C₆ alkoxy group or a (C₁-C₆ alkyl) carbonyloxy group,
n is an integer between 8 and 20,
or the pharmaceutically acceptable addition salts, isomers, enantiomers or diastereoisomers thereof, as well as mixtures thereof.

2. The compound according to claim 1, wherein R₁, R₂, and R₃ represent a methyl group or a hydrogen atom.

3. The compound according to claim 1, wherein R₄, R₅, R₆ or R₇ represent a hydrogen atom or a methoxy group.

4. The compound according to claim 1, wherein R₅, R₆ and R₇ represent a hydrogen atom.

5. The compound according to claim 1, wherein n is an integer equal to 10, 12, 14, 16, or 18.

6. The compound according to claim 1, wherein n is an integer equal to 10 or 12.

7. The compound according to claim 1, wherein said compound is selected from the group consisting of:
RFA-12 of the formula below: 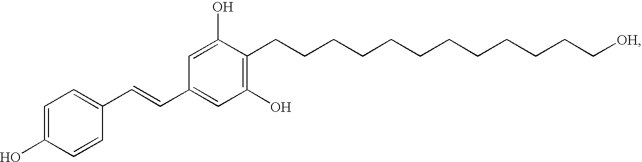
MRFA-12 of the formula below: 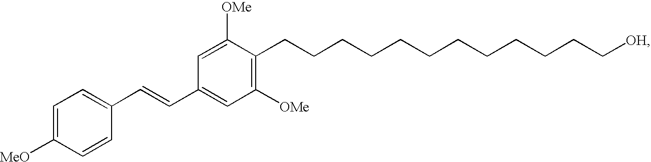
DMRFA-12 of the formula below: 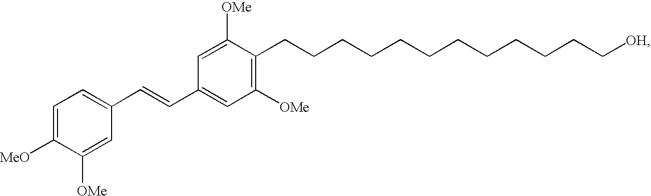
MRFA-10 of the formula below: 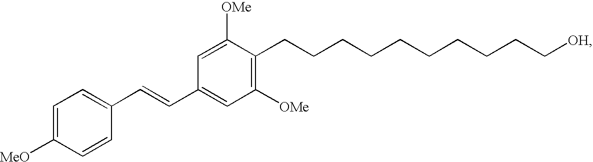
and DMRFA-10 of the formula below:

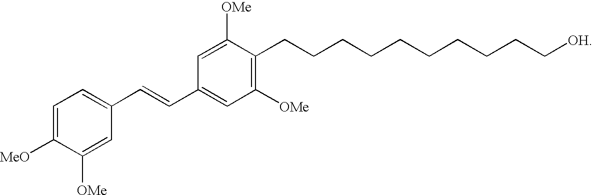

8. The compound according to claim 7, wherein said compound is RFA-12.

9. A pharmaceutical composition which comprises at least one compound according to claim 1 in combination with a pharmaceutically acceptable excipient.

10. (canceled)

11. A method for preventing or treating nervous system diseases or disorders that alter neurons or other cells of the nervous system and/or diseases or disorders of nervous system inflammation, and/or degenerative neuropathies, and/or demyelinating or dysmyelinating disorders and/or cerebral vascular accidents or any other lesional attacks of the nervous system comprising the administration of an effective amount of a compound according to claim 1 or of a pharmaceutical composition according to claim 9 to a patient in need thereof.

12. The method according to claim 11, wherein the degenerative neuropathies are multiple sclerosis, Alzheimer's disease, Parkinson's disease or Creutzfeldt-Jakob disease.

13. A method for modulating the cellular specification of neural stem cells, favoring the differentiation and then the survival of the neurons and glial cells in differentiation, favoring the differentiation of oligodendrocyte precursors cells into mature oligodendrocytes, and/or for decreasing the activation of microglia and/or the activation of astrocytes and/or the reactive gliosis comprising the administration of an effective amount of a compound according to claim 1 or a pharmaceutical composition according to claim 9 to a patient in need thereof.

14. A method for the in vitro obtention of neurons and/or glial cells differentiated from stem cells comprising the administration of an effective amount of at least one compound according to claim 1 to said stem cells.

15. A method for preparing a compound of general formula (I) according to claim 1 wherein R₁, R₂ and R₃ represent a hydrogen atom wherein said method comprises step (a) of reacting the compound of general formula (I), wherein R₁, R₂ and R₃ represent a C₁-C₆ alkyl group, with boron tribromide in dichloromethane at −78° C.

16. A method for preparing a compound of formula (I) according to claim 1 wherein R₁, R₂ and R₃ represent a C₁-C₆ alkyl group wherein it comprises step (b) of Wadsworth-Emmons coupling between the compound of general formula (II) below: 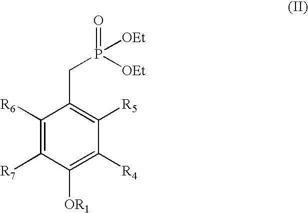
wherein R₁ represents a C₁-C₆ alkyl group and R₄ is as defined in claim 1, and the compound of general formula (III) below: 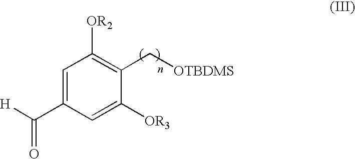
wherein R₂ and R₃ represent a C₁-C₆ alkyl group and n is as defined in claim.

17. The method according to claim 16 wherein the compound of general formula (III) is obtained by steps (c), (d) and (e) below:
(e) catalytic hydrogenation of the compound of general formula (IV) below: 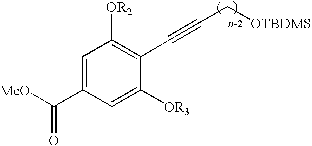
wherein R₂ and R₃ represent a C₁-C₆ alkyl group, and n is as defined in claim 16, to obtain the compound of general formula (V) below: 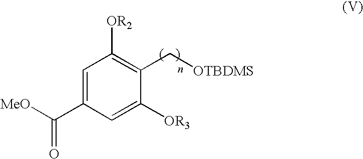
wherein R₂ and R₃ represent a C₁-C₆ alkyl group, and n is as defined in claim 16,
(d) reduction of the ester of formula (V) into the alcohol of general formula (VI) below: 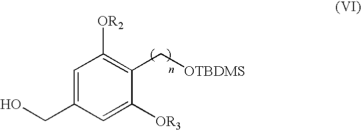
wherein R₂ and R₃ represent a C₁-C₆ alkyl group, and n is as defined in claim 16
(c) oxidation of the alcohol of formula (VI) into the aldehyde of formula (III)

18. The method according to claim 17, wherein the compound of general formula (IV) is obtained by step (f) of the Sonogashira coupling reaction between the compound of general formula (VII) below: 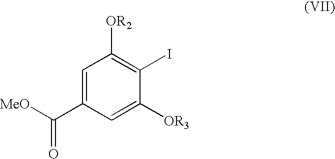
wherein R₂ and R₃ represent a C₁-C₆ alkyl group, and the compound of general formula (VIII) below: 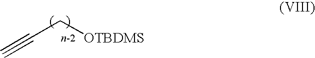
wherein n is as defined in claim 17.

19. The method according to claim 16, wherein R₁, R₂ and R₃ represent a methyl group and R₄ represents a hydrogen atom.
